(19) United States
(12) Patent Application Publication
(10) Pub. No.: US 2022/0000534 A1
(43) Pub. Date: Jan. 6, 2022

(54) SURGICAL TOOL

(71) Applicant: BEIJING SURGERII TECHNOLOGY CO., LTD., Beijing (CN)

(72) Inventors: Kai XU, Beijing (CN); Yitang REN, Beijing (CN)

(21) Appl. No.: 17/292,548

(22) PCT Filed: Dec. 27, 2019
(86) PCT No.: PCT/CN2019/128984

§ 371 Date: May 10, 2021

(30) Foreign Application Priority Data
Dec. 27, 2018 (CN) 201811612725.0

(51) Int. Cl. A61B 18/08; A61B 18/14

(52) U.S. Cl. CPC A61B 18/085; A61B 2018/126; A61B 18/1492; A61B 18/1445

(57) ABSTRACT

Disclosed is a surgical tool. The surgical tool comprises a surgical tool head (11) and a rod shaft (12). The surgical tool head (11) is connected with the shaft (12). The shaft (12) is a flexible shaft and comprises a flexible core and a flexible sleeve. The flexible sleeve has an inner cavity, and the flexible core is slidably disposed in the inner cavity. The surgical tool head (11) comprises two jaws (111, 112). The flexible core and the flexible sleeve are respectively connected with the two jaws (111, 112), and the flexible core is able to slide relatively to the flexible sleeve to drive the two jaws (111, 112) to make relative opening and closing movements. The shaft (12) of the surgical tool can be bent freely into any shape, and thus can be integrated into a flexible surgical tool and applied in a wider range.

### TECHNICAL FIELD

The present disclosure relates to the field of medical instrument, in particular to a surgical tool.

### BACKGROUND

Surgical forceps are commonly used for more delicate operations such as brain surgery, microsurgery, ENT (ear, nose and throat), obstetrics and gynecology, and hand surgery, and their use is gradually expanding. The prior art surgical forceps include a surgical tool head, a rod in the middle, and an actuator. The surgical tool head and the actuator are connected by the rod in the middle. The tail device in the prior art has a rigid structure and cannot be bent freely. Therefore, it cannot be integrated into a flexible surgical tool, which limits its use range.

Therefore, there is a need in the art for a surgical tool that can be flexible.

### SUMMARY

An object of the present disclosure is to provide a surgical tool, comprising: a surgical tool head and a shaft, wherein the surgical tool head is connected with the shaft, and the shaft is a flexible shaft and comprises: a flexible core; a flexible sleeve, the flexible sleeve having an inner cavity, and the flexible core being slidably disposed in the inner cavity; wherein the surgical tool head comprises two jaws, the flexible core and the flexible sleeve are respectively connected with the two jaws, and the flexible core is able to slide relatively to the flexible sleeve to drive the two jaws to make relative opening and closing movements.

In a preferable embodiment, the flexible core comprises: an elastic metal wire; a first covering tube wrapping the elastic metal wire and fixed relatively to the elastic metal wire.

In a preferable embodiment, the flexible sleeve comprises: a flexible metal tube forming the inner cavity; and a second covering tube wrapping the metal tube and fixed relatively to the metal tube.

In a preferable embodiment, the two jaws are two electrodes, and the two electrodes are connected with the metal tube and the elastic metal wire, respectively.

In a preferable embodiment, the metal tube is formed of a spiral metal band, a metal wire mesh or a corrugated metal tube.

In a preferable embodiment, the elastic metal wire is made of nickel-titanium alloy.

In a preferable embodiment, the first covering tube is made of wear-resistant resin.

In a preferable embodiment, the second covering tube is made of resin or heat shrinkable tube.

In a preferable embodiment, the first covering tube is made of polytetrafluoroethylene.

In a preferable embodiment, the flexible sleeve extends to inside of the surgical tool head.

Compared with the prior art, the shaft of the surgical tool according to the present disclosure can be freely bent to any shape, and thus can be integrated into a flexible surgical tool and applied to a wider range during surgery.

### BRIEF DESCRIPTION OF THE DRAWINGS

FIG. 1 is a schematic diagram of an embodiment of a surgical tool according to the present disclosure.

FIG. 2 is a perspective view of an embodiment of a surgical tool according to the present disclosure.

FIG. 3 is a half-cut perspective view of a surgical tool according to the present disclosure.

FIG. 4 is a schematic diagram of another embodiment of a surgical tool according to the present disclosure.

FIG. 5 is a perspective view of another embodiment of a surgical tool according to the present disclosure.

FIG. 6 is an internal structure diagram of a surgical tool head according to another embodiment of the surgical tool of the present disclosure.

### DETAILED DESCRIPTION OF EMBODIMENTS

Hereinafter, the preferred embodiments of the present disclosure will be described in detail with reference to the accompanying drawings, so as to better understand objects, features and advantages of the present disclosure. It can be understood that the embodiments shown in the drawings are not intended to limit the scope of the present disclosure, but merely to illustrate an essential spirit of technical solutions of the present disclosure.

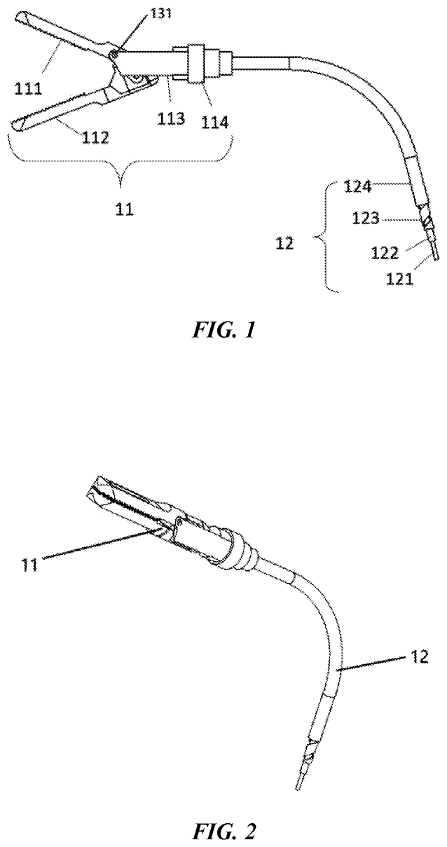

FIG. 1 is a schematic diagram of a surgical tool head 11 and a shaft 12 of an embodiment of a surgical tool according to the present disclosure. The shaft 12 is a flexible rod and can be bent freely. The surgical tool head 11 includes a first jaw 111, a second jaw 112, a base 113 and an insulator 114. The shaft 12 has a multi-layer nested structure. The shaft 12 has a flexible core. In the illustrated embodiment, the flexible core is a superelastic metal wire 121, and a first covering tube 122 is wrapped around the superelastic metal wire 121. The first covering tube 122 tightly wraps the superelastic metal wire 121 and is fixed relatively to the superelastic metal wire 121. However, it can be understood that the flexible core of the present disclosure is not limited to this, and any core that is flexible and can be bent falls within the scope of the present disclosure. A sleeve is provided outside a first covering tube 122. The sleeve has an inner cavity. The sleeve can include a metal tube 123 and a second covering tube 124, and the superelastic metal wire 121 and the first covering tube 122 can be slidably disposed in an inner cavity of the metal tube 123. In the illustrated embodiment, the metal tube 123 forms an inner cavity, and the superelastic metal wire 121 and the first covering tube 122 are slidably disposed in the inner cavity of the metal tube 123. Second covering tube 124 is wrapped on the outside of the metal tube 123. Similarly, the second covering tube 124 tightly wraps the metal tube 123 and is fixed relatively to the metal tube 123, thereby isolating the metal tube 123 from the outside. In such a structure, the superelastic metal wire 121 and the metal tube 123 are respectively connected with two jaws 111 and 112 of the surgical tool head 11. The superelastic metal wire 121 and the first covering tube 122 can slide reciprocatingly in the inner cavity of the metal tube 123 with respect to the metal tube 123 and the second covering tube 124. The reciprocating sliding can make the two jaws of the tool head 11 open and close. The movement of the superelastic metal wire 121 and the first covering tube 122 relative to the metal tube 123 and the second covering tube 124 can be realized by an actuator (not shown) connected with the shaft 12. The actuator may be in various forms, such as a handle of a scissor structure or any other device that enables the flexible core to slide relative to the flexible sleeve.

In the embodiment shown in FIG. 1, the two forceps 111 and 112 may be respectively formed as having electrodes, and the two electrodes are respectively electrically connected with the above-mentioned superelastic metal wire 121 and the metal tube 123. Therefore, the two electrodes can be supplied with power through the superelastic metal wire 121 and the metal tube 123. As such, the surgical tool can be used as a bipolar coagulation forceps to achieve blood coagulation and other functions. The first covering tube 122 and the second covering tube 124 can be used as insulation layers to electrically insulate the superelastic metal wire 121 and the metal tube 123 and the metal tube 123 from the outside, respectively.

The metal tube 123 also has a supporting function, which can keep the inner cavity from collapsing and maintain the gap between the metal tube 123 and the first covering tube 122, so as to prevent reciprocating sliding of the superelastic metal wire 121 and the first covering tube 122 from pulling the metal tube 123, especially in the case that the metal tube 123 is made of spiral metal band and metal wire mesh. The metal tube 123 may be in various forms as long as the metal tube 123 can be easily bent. For example, as shown in FIG. 1, the metal tube 123 is in a form of a spiral metal band. The metal tube 123 may also be formed of a metal wire mesh or a corrugated metal tube.

Preferably, a gap is formed between the first covering tube 122 and the metal tube 123, and the gap is preferably uniform along a length of the shaft 12 to prevent movement resistance of the superelastic metal wire 121 and the first covering tube 122 from increasing due to compression from the outside. In addition, the inner cavity of the metal tube 123 extends to the inside of the surgical tool head 11. Since the surgical tool is a medical instrument, it has a strict sterilization procedure. Therefore, the surgical tool can be sterilized more effectively by injecting water and disinfectant into the inner cavity.

The superelastic metal wire 121 may be made of any suitable metal. Preferably, the superelastic metal wire 121 is made of nickel-titanium alloy, which can be bent in any direction, and the nickel-titanium alloy has good memory ability, which facilitates the restoration of the shaft 12 to its original shape. The first covering tube 122 is usually made of a wear-resistant resin to withstand the reciprocating movement relative to the metal tube 123, thereby extending the life of the surgical tool. Preferably, the first covering tube 122 is made of polytetrafluoroethylene. A smooth surface of a polytetrafluoroethylene tube and its non-stick type can reduce the resistance of liquid flow, reduce the attachment of bacterium plaques, and improve the disinfection effect.

The second covering tube 124 is preferably made of resin. To facilitate manufacturing, a heat shrinkable tube may also be used to form the second covering tube 124.

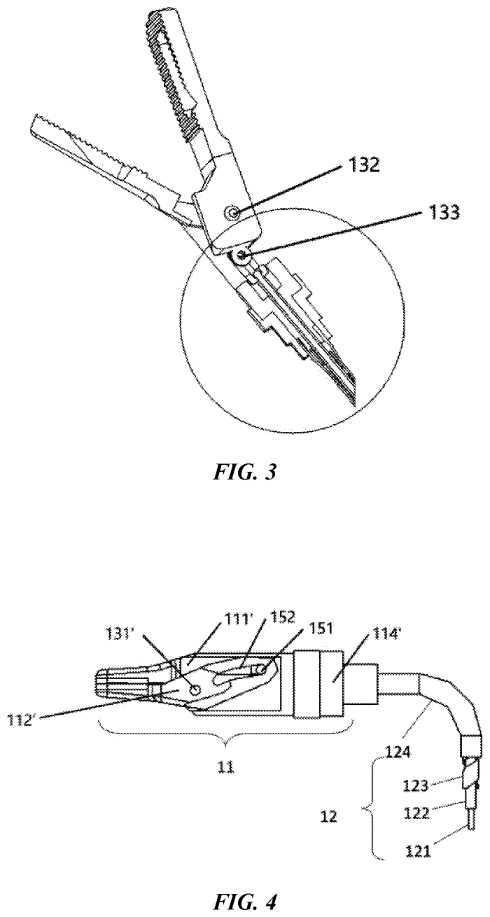

In the embodiment shown in FIG. 1, the surgical tool head 11 includes an insulator 114, a base 113 and two jaws 111 and 112. The shaft 12 is connected with the two electrodes and passes through the insulator 114. The base 113 extends from the insulator 114 toward the jaws. In the illustrated embodiment, the base 113 includes two extension parts extending from opposite positions at outer periphery of the insulator 114. The first jaw 111 is pivotally connected with the base 113 by a pivot 131, and as shown in FIG. 3, the second jaw 112 is pivotally connected with the jaw 111 by a pivot 132 at a position closer to the insulator 114 than the pivot 131. The second jaw 112 is pivotally connected with the superelastic metal wire 121 at an end close to the insulator 114 by a pivot 133. Therefore, when the superelastic metal wire 121 and the first insulating tube 122 reciprocatingly slide in the metal tube 123, the second jaw 112 is driven to pivot around the pivot 132 relatively to the first jaw 111, and the first jaw 111 is driven to pivot around the pivot 131, thereby realizing opening and closing of the two jaws 111 and 112. In addition, in the case where the two jaws 111 and 112 are used as electrodes, an insulating sleeve may be provided on outer surface of a pivot pin at the pivot 132 to ensure insulation between the two electrodes.

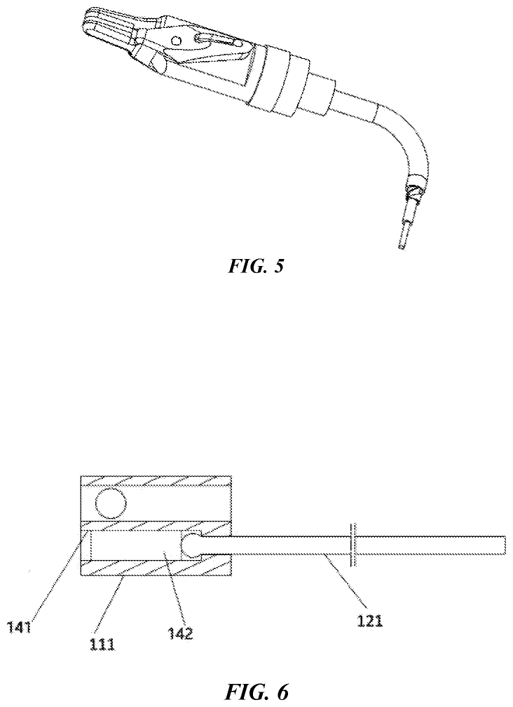

FIGS. 4 and 5 respectively show a schematic diagram and a perspective view of a surgical tool head 11 and a shaft 12 of a surgical tool according to a second embodiment of the present disclosure. The structure of the shaft 12 is the same as the above-mentioned embodiment. The surgical tool head 11 includes an insulator 114′, two jaws 111′ and 112′. The first jaw 111′ is fixed to the insulator 114′, and the second jaw 112′ has a “Y” shape and is pivotally connected with the first jaw 111′ at a pivot 131′. The pivot 131′ and the insulators 114′ are separated by a certain distance. As shown in FIG. 6, an axial sliding channel 141 is provided in the first jaw 111′, and the superelastic metal wire 121 is connected with the slider 142 in the sliding channel, so that sliding of the superelastic metal wire 121 and the first insulating tube 122 in the metal tube 123 drives the slider 142 to slide in the sliding channel 141. In addition, protrusions 151 are provided on both sides of the slider 142. The jaw 112′ has two branch parts close to the insulator 114′. The two branch parts are provided with arc-shaped sliding grooves 152. The protrusions 151 can slide in the sliding grooves 152. Therefore, sliding of the slider 142 and the protrusions 151 through the sliding grooves 152 drives the second jaw 112′ to pivot relatively to the first jaw 111′, thereby achieving opening and closing between the two jaws. In addition, in the case where the two jaws 111 and 112 are used as electrodes, an insulating sleeve may be provided on outer surface of a pivot pin at 131′ to ensure insulation between the two electrodes.

It can be understood that the present disclosure is not limited to the above two embodiments, but can be applied to surgical tools of other structures without departing from the scope of the present disclosure.

The preferred embodiments of the present disclosure have been described in detail above, but it can be understood that after reading the above content of the present disclosure, those skilled in the art can make various changes or modifications to the present disclosure. These equivalents also fall within the scope defined by the appended claims of this application.

1. A surgical tool, comprising:
a surgical tool head comprising a first jaw and a second jaw; and
a shaft connected with the surgical tool head and comprising:
a flexible core connected with the second jaw; and
a flexible sleeve connected with the first jaw, the flexible sleeve comprising an inner cavity, and the flexible core being slidably disposed in the inner cavity and operable to slide relatively to the flexible sleeve to drive relative opening and closing of the first and second jaws.

2. The surgical tool according to claim 1, wherein the flexible core comprises:
an elastic metal wire;
a first covering tube wrapping the elastic metal wire and fixed relatively to the elastic metal wire.

3. The surgical tool according to claim 1, wherein the flexible sleeve comprises:
a metal tube forming the inner cavity; and
a second covering tube wrapping the metal tube and fixed relatively to the metal tube.

4. The surgical tool according to claim 3, wherein the first jaw comprises an electrode connected with the metal tube.

5. The surgical tool according to claim 3, wherein the metal tube comprises a spiral metal band, a metal wire mesh or a corrugated metal tube.

6. The surgical tool according to claim 2, wherein the elastic metal wire comprises nickel-titanium alloy.

7. The surgical tool according to claim 2, wherein the first covering tube comprises wear-resistant resin.

8. The surgical tool according to claim 3, wherein the second covering tube comprises resin or heat shrinkable tube.

9. The surgical tool according to claim 2, wherein the first covering tube comprises polytetrafluoroethylene.

10. The surgical tool according to claim 1, wherein the flexible sleeve extends to inside of the surgical tool head.

11. The surgical tool according to claim 2, wherein the second jaw comprises an electrode connected with the elastic metal wire.

12. The surgical tool according to claim 2, wherein the elastic metal wire comprises a block at an end close to the surgical tool head.

13. The surgical tool according to claim 1, further comprising a base, wherein the first jaw is pivotally connected with the base by a first pivot.

14. The surgical tool according to claim 13, wherein the first jaw is pivotally connected with the second jaw by a second pivot.

15. The surgical tool according to claim 14, wherein the second jaw is pivotally connected with the flexible core by a third pivot.

16. The surgical tool according to claim 14, further comprising an insulating sleeve on outer surface of the second pivot.

17. The surgical tool according to claim 1, further comprising an insulator, wherein the first jaw is fixed to the insulator, and the second jaw is pivotally connected with the first jaw by a fourth pivot.

18. The surgical tool according to claim 17, further comprising:
a slider connected with the flexible core and slidable in an axial sliding channel on the first jaw.

19. The surgical tool according to claim 18, wherein the second jaw comprises a first branch part having a first arc-shaped sliding groove and a second branch part having a second arc-shaped sliding groove, and
the slider comprises a first protrusion slidable in the first arc-shaped sliding groove and a second protrusion slidable in the second arc-shaped sliding groove.

20. The surgical tool according to claim 17, further comprising an insulating sleeve on outer surface of the fourth pivot.
